(19) United States
(12) Patent Application Publication
(10) Pub. No.: US 2022/0304890 A1
(43) Pub. Date: Sep. 29, 2022

(54) REAL TIME CARDIOPULMONARY RESUSCITATION (CPR) FEEDBACK WITH INSTRUCTIONS APPARATUS AND METHOD OF USE

(71) Applicants: Abigail Lynn KOHLER, Newtown, CT (US); Gregory BOUDREAU-FINE, Rehoboth, MA (US)

(72) Inventors: Abigail Lynn KOHLER, Newtown, CT (US); Gregory BOUDREAU-FINE, Rehoboth, MA (US)

(21) Appl. No.: 17/616,171

(22) PCT Filed: Jun. 3, 2020
(86) PCT No.: PCT/US2020/035991

§ 371 Date: Dec. 2, 2021

Related U.S. Application Data
(60) Provisional application No. 62/856,544, filed on Jun. 3, 2019.
(60) Provisional application No. 62/972,574, filed on Feb. 10, 2020.
(60) Provisional application No. 62/972,544, filed on Feb. 10, 2020.

(51) Int. Cl. A61H 31/00; G09B 23/28

(52) U.S. Cl. CPC A61H 31/005; A61H 31/007; G09B 23/288; A61H 2201/5064; A61H 2201/165; A61H 2201/1638; A61H 2201/5097; A61H 2201/5043; A61H 2201/5084; A61H 2201/5007; A61H 2201/0107; A61N 1/39044

(57) ABSTRACT

A device for first responders and medical professionals to assist during the administration of cardiopulmonary resuscitation (CPR) on a patient in need thereof. The device may also be used to train individuals on how to properly perform cardiopulmonary resuscitation by providing initial instructions about the process as well as real-time monitoring of the student's technique and providing real-time instructive feedback to the student so that the student may improve their skills. The invention also includes a method of using the device to coach first responders and medical professionals as they perform cardiopulmonary resuscitation (CPR) on a patient in need thereof as well as a method of using the device to teach students about cardiopulmonary resuscitation (CPR).

### CROSS REFERENCE TO RELATED APPLICATIONS

This application claims priority to and the benefit of U.S. Provisional Patent Application No. 62/972,574 filed Feb. 10, 2020, U.S. Provisional Patent Application No. 62/972,544 also filed on Feb. 10, 2020, and U.S. Provisional Patent Application No. 62/856,544 filed Jun. 3, 2019, the entire contents of each are incorporated by reference herein.

### FIELD OF THE INVENTION

This invention relates to the field of medical devices. More specifically, it relates to coaching and assistive devices used by first responders, medical professionals, and other rescuers while performing cardiopulmonary resuscitation (CPR) or learning to do so.

### BACKGROUND OF THE INVENTION

Chest compressions are an important part or CPR where the rescuer or first responder places one hand on top of the other and pushes on the victim's chest, ideally at a rate and force in accordance with medical guidelines, e.g., the American Heart Association® (AHA) guidelines (Virani S S et al., (2020), Circulation, 141(9):e139-56). The goal of these compressions is to maintain blood flow and oxygen supply to the victim's body when their heart is beating irregularly or not at all. It is important for the rescuer or first responder to apply compressions with enough force and frequency to create adequate blood circulation for the victim. When done correctly, CRP can increase the likelihood of the victim's survival.

Administering CPR correctly, however, can be difficult. Rescuers or first responders or first responders often have to perform chest compressions in stressful situations and for extended periods of time. The rescuer or first responder can become fatigued or have their focus impaired. Under these conditions, it is very difficult to effectively estimate the force that needs to be applied to the victim's chest or the frequency of compressions required to give the victim proper blood circulation. Studies have demonstrated that even trained professionals often misjudge these two parameters while performing CPR and, as a result, provide less than adequate CPR for the victim, hurting their odds of survival.

For this reason, there is a need for a practical device that can measure various parameters of the rescuer's or first responder's CPR performance and give feedback in an effective way, in real time. This would be useful in real medical emergencies or for practicing CPR in a training setting. Devices have been proposed to help with this. One instance of this is U.S. Pat. No. 5,496,257 (Kelley) that discloses a device placed on the victim's chest and uses a pressure sensor to measure compression forces and timing. The device has a visual and audio feedback system built into the same housing that holds the pressure sensors. This could make the device difficult to use in certain conditions such as the back of a moving ambulance because the device would not be secured in place. The device would be free to move anytime the rescuer or first responder is not actively holding it in place, for example, while they are delivering rescue breaths between compressions. Additionally, it is standard to be trained to perform CPR wearing only light gloves so adding a bulky housing between the rescuer's or first responder's hands and the patient's chest could be unfamiliar or uncomfortable for the rescuer or first responder.

Another instance of a CPR assistive device is described in U.S. Pat. No. 9,028,259 (Centen et al.) that discloses a wearable device that goes on one of the rescuer's or first responder's hands to measure CPR parameters. To display visual feedback to the user, the patent describes transmitting the data “to a separate computing device, such as a personal computer or a portable wireless device for display.” This is not desirable because the separate computing device would draw the rescuer's or first responder's attention away from the victim. Even if the separate device is moved to be proximate to the victim and site of compression, it could add unneeded complexity to the system or be unstable if used, for example, in a moving ambulance. The patent also describes an alternative apparatus where feedback is displayed on the back of the hand wearing the glove. This would not work well because the rescuer or first responder needs to place one hand over the other while performing CPR. The back of the hand with the sensors and display would be obstructed by the other hand.

Another instance of a CPR assistive device is described in U.S. Pat. No. 8,147,433 (Halperin et al.) that discloses a CPR-assistive device that uses an accelerometer in a location fixed to the patient's chest to measure compression depth. It determines depth of compression independently of any reference data. This system is not desirable due to the absence of reference data indicating movements of the patient's body not caused by chest compressions. If the device were to be used in a moving vehicle like an ambulance, the device might not be able to discern movements of the vehicle from movement caused by chest compression. Taking into consideration possible data filtering methods, noise still impacts the accuracy of the device. Using an additional reference device such as an accelerometer would allow the device's processor to more effectively differentiate chest compression movement from other movements of the victim's body even when the movements have the same frequency and share other characteristics.

Another instance of a CPR assistive device is described in U.S. Pat. No. 9,585,603 (Centen). It discloses a CPR assistive device that uses “a field generator, a field detector, and a processor” to determine the depth of chest compressions during CPR. The field generator acts as a reference to move with the patient's body so the field detector will only measure motion about this reference generator. This would be an adequate way to differentiate chest compression movements from movements of the patient's body. This is not desirable, however, because any type of electric or magnetic field used in this way could interfere with a patient's pacemaker or other implanted metal or electronic devices.

### BRIEF SUMMARY OF THE INVENTION

The present invention is a medical device to assist a rescuer or a first responders or first responders in performing CPR more effectively by giving real time feedback on the quality of compressions and/or how the compressions should be corrected. The device can also be used in the same way while a student (person learning or practicing CPR) is practicing CPR chest compressions. The device will include one or more sensors to detect one or more parameters relating to the quality of the rescuer's or first responder's CPR chest compressions. These sensors can be positioned between the rescuer's or first responder's lower hand and the victim's chest, on the back of the rescuer's or first responder's upper hand, or at any other position adequate for the sensor's detection.

The device will also include a display or other feedback system. This system will provide instructions pertaining to CPR. This system will also be used to provide visual CPR feedback or queues for performing better CPR. The device may also include auditory and/or tactile outputs to go along with or replace the visual display system. Using the described device and method will allow a rescuer or first responder to provide the best possible care when performing CPR, giving the optimal survival probability to the victim.

### BRIEF DESCRIPTION OF THE FIGURES

FIG. 1 is an illustration depicting a rescuer or first responder 11 administering CPR to a victim 14.

FIG. 2 is a schematic drawing of from the perspective of the top of the main device 12 as shown in FIG. 1.

FIG. 3 illustrates an exploded view of the main device 12 as shown in FIG. 1.

FIG. 4 illustrates an exploded view of the adhesive component.

FIG. 5 shows an exploded view of the reference device 13.

FIG. 6 is a flow chart describing the data processing pathway.

FIG. 7 illustrates the container or hub 71 that is used as a central location to store the device.

FIG. 8 depicts the rescuer or first responder 11 administering CPR to the victim 14.

FIG. 9 depicts the hardware components of the device.

FIG. 10 depicts the process by which the system could be used to provide instructions before CPR and feedback during CPR in order to guide the user in the proper administration of CPR.

FIG. 11 depicts the process by which the algorithm processes and analyzes the data from the accelerometer, compares it to a standard and provides feedback to the rescuer or first responder. This process repeats as long as the rescuer or first responder is performing CPR.

FIG. 12 depicts a student 11 practicing CPR on a mannequin 13. An embodiment of the CPR assistive device 12 described herein is attached to the back of the student's top hand 11 while practicing CPR. In some preferred embodiments, the CPR assistive device can be a smart phone 11 which includes appropriate functions described herein.

FIG. 13 depicts the process by which the system is used in order to train or practice the proper administration of CPR.

FIG. 14 demonstrates how a camera could be used in order to train or practice the proper administration of CPR.

FIG. 15 depicts the process by which a mannequin or dummy would be sent by an instructor and received by a student in order to train or practice the proper administration of CPR.

FIG. 16 depicts the process by which use of a generated authentication key unlocks and locks the software.

FIG. 17 demonstrates the process through which the software is locked and unlocked with the use of a static authentication key.

### DETAILED DESCRIPTION OF THE INVENTION

Disclosed herein is a novel device and method to assist rescuers or first responders in the performance of CPR or to assist a student in learning or practicing CPR.

### Apparatus

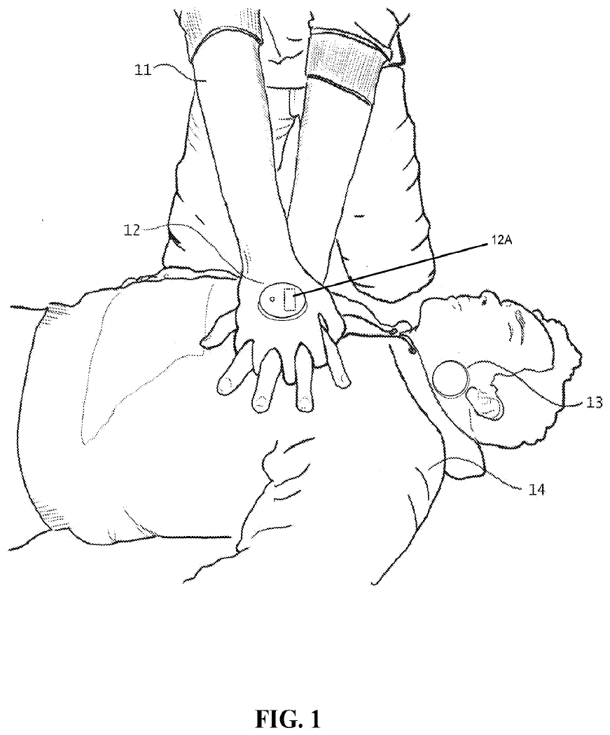

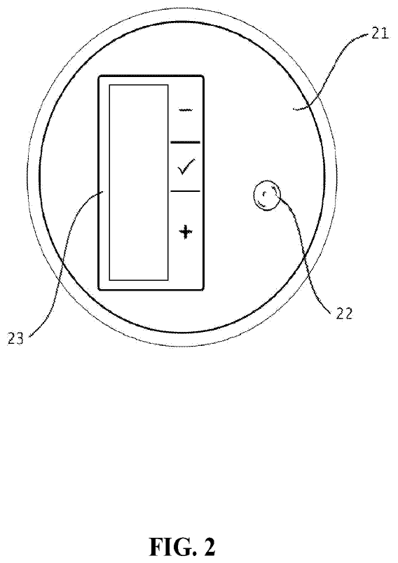

FIG. 1 shows an embodiment of the invention which includes two parts: a main device 12 and a reference device 13 (collectively referred to hereinafter as “the device”). The main device 21 is comprised of a housing made from a semiflexible polymer selected from the group, including, but not limited to polyvinyl chloride (PVC), polypropylene (PP), polyethylene (PE), polystyrene (PS) as well as nylon, polyethylene terephthalate (PET), polyimide (PA), polycarbonate (PC), acrylonitrile butadiene (ABS), polyurethane (PU) and polyetheretherketone (PEEK) (BMP Medical, Sterling, Mass.) with a non-conductive adhesive (or medically-approved adhesive pads that may be replaced and discarded after use) (Panacol-USA, Torrington, Conn.) on one side (not shown) and a user feedback display 12A on the opposite side. When preparing to perform CPR, the main device 12 will be adhered to the back of the rescuer's or first responder's hand 11 or glove (not shown) or held by the rescuer or first responder. As shown in FIG. 1, it can be adhered (disposable adherence pad not shown) to the back of the hand 11 or glove that will be on top when the rescuer's or first responder's hands 11 are placed in position to perform CPR. The device 12 will be able to use data from an accelerometer, and possibly other sensors, to measure the depth of displacement of the rescuer's or first responder's hands as they perform CPR chest compressions. Based on the depth of displacement, the device 12 will give visual or other feedback, as shown in FIG. 2 (the top view of the main device 21), to the rescuer or first responder 11 so they can apply more or less force with each compression, as necessary. The rescuer or first responder 11 would be able to see the compression depth displayed on a continuous monitor 23 that has marks, i.e., “−”, “4” or “+” indicating whether the depth was too little, adequate or too much. There would also be a frequency indicator 22 that would consist of either a vibration motor, blinking diode, or speaker 25 that would pulse at the correct compression pace.

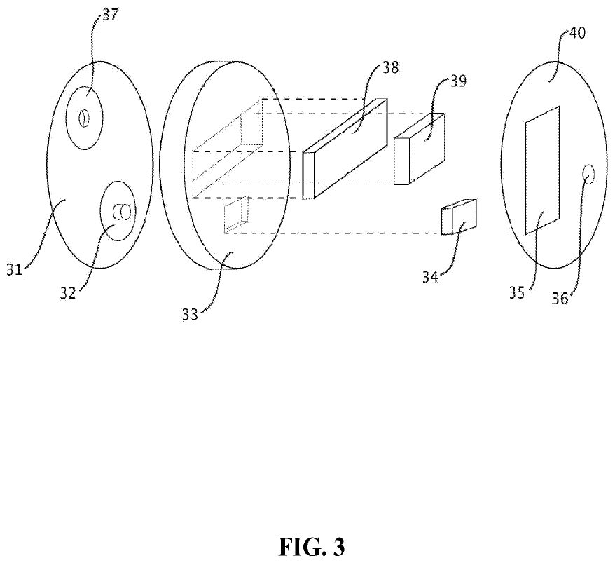

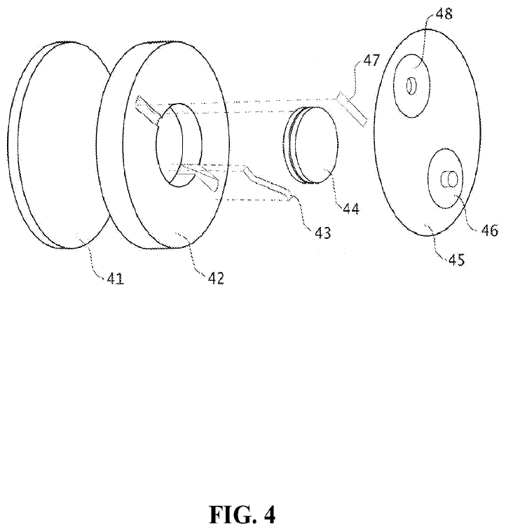

Each main device 12, as shown in FIGS. 3 and 4, comprises a housing 33 containing a power source 44 such as a CR1620 (Panasonic®, Kadoma-shi, Osaka, JP) or other watch batteries (Energizer® Holdings, St. Louis, Mo.), an accelerometer 39 such as the LIS3DH triple-axis accelerometer (Adafruit Industries LLC, New York, N.Y.), a processor 38 such as an ATMEGA32U4-AU (AVR AVR® ATmega Microcontroller IC 8-Bit 16 MHz 32 KB (16K×16) FLASH 44-TQFP (10×10)(Microchip Technology, Inc., Chandler, Ariz.), and a feedback display 35 such as a Nokia 5110/3310 monochrome LCD (Nokia®, Espoo, FI). It may also include one or more vibration motors 36 (Adafruit® Industries LLC, New York, N.Y.) and/or a wired or wireless data transmission system 34. There is a non-conductive adhesive layer 41 (Panacol-USA, Torrington, Conn.) attached to housing 42 for the battery 44. The battery 44 is connected to conductive strips 43 and 47 that carry current to snaps 46 and 48 that are embedded into the top layer of the device 45. A non-conductive adhesive pad 31 comprising snaps 32 and 37 embedded in the non-conductive adhesive pad 31 connect the electronics to the battery 44. The device has a top cover 40 that contains a screen 35 and a vibration motor, blinking diode or speaker 36.

The main device 12 is paired with a reference device 13 shown in FIG. 1. As depicted, the main device 12 is adhered to the back of the rescuer's or first responder's 11 top hand while performing CPR. The reference device 13 is adhered to the victim's 14 neck. The reference device 13 is also comprised of a housing made from a semiflexible polymer selected from the group, including, but not limited to polyvinyl chloride (PVC), polypropylene (PP), polyethylene (PE), polystyrene (PS) as well as nylon, polyethylene terephthalate (PET), polyimide (PA), polycarbonate (PC), acrylonitrile butadiene (ABS), polyurethane (PU) and polyetheretherketone (PEEK) (BMP Medical, Sterling, Mass.) with a non-conductive adhesive (or medically-approved adhesive pad that may be replaced and discarded after use) (Panacol-USA, Torrington, Conn.) on one side (not shown). As the rescuer or first responder 11 is preparing to perform CPR on the victim 14, the reference device 13 is affixed to the side of the victim's 14 neck, the victim's 14 vertebro-distal rib near the 6^{th} intercostal space, the victim's 14 back, or on another stable part of the victim's 14 body using the non-conductive adhesive (or medically-approved adhesive pad that may be replaced and discarded after use) (Panacol-USA, Torrington, Conn.). The reference device 13 measures movement of the victim 14 so it should be placed on part of the victim 14 that moves with the torso of the victim 14, but independently of the chest compressions administered to the victim 14.

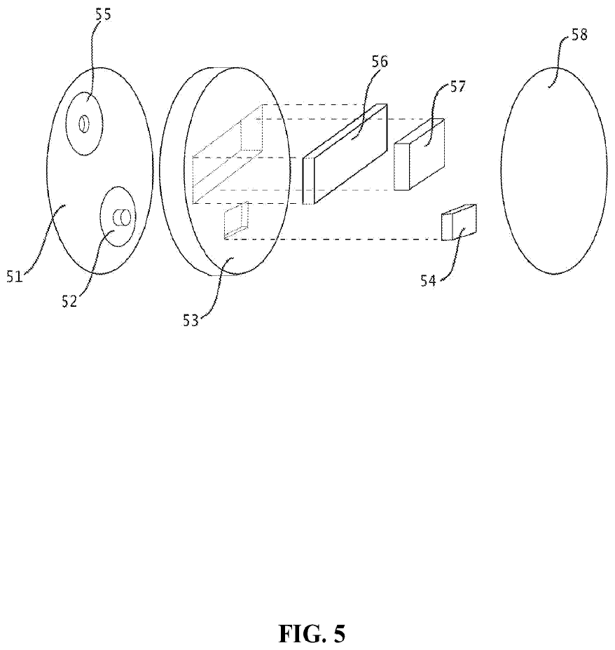

The reference device 13, as shown in FIGS. 4 and 5 includes an non-conductive adhesive pad 51 (Panacol-USA, Torrington, Conn.), power source such as a CR1620 (Panasonic®, Kadoma-shi, Osaka, JP) or other watch batteries (Energizer® Holdings, St. Louis, Mo.), an accelerometer 57 such as the LIS3DH triple-axis accelerometer (Adafruit® Industries LLC, New York, N.Y.), a processor 56 such as an ATMEGA32U4-AU (AVR AVR® ATmega Microcontroller IC 8-Bit 16 MHz 32 KB (16K×16) FLASH 44-TQFP (10×10)(Microchip Technology, Inc., Chandler, Ariz., and a wired or wireless data transmission system. The reference device 13 includes a layer that connects the adhesive pad 51 using snaps 52 and 55 that connect the electronics to the battery (not shown). There is also housing 53 in which the electronics would be embedded. The device also includes a top cover 58.

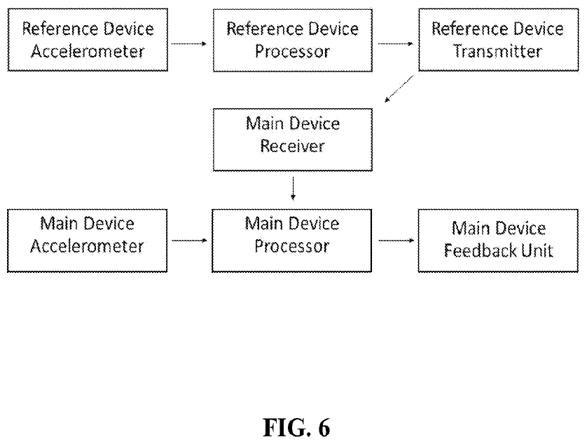

The device will operate as shown in FIG. 6 that depicts how data from both the reference device 13 and main device 12 is transmitted to the main device 12. When the main device 12 is turned on, the accelerometer 39 in the main device 12 and the accelerometer 57 in the reference device 13 will begin collecting data. The data from the main device's accelerometer 39 will be transmitted to the processor 38 in the main device 12. Simultaneously, the data from the reference device's 13 accelerometer 57 will be sent to the reference device's 13 processor 56. The data from the reference device's 13 processor 56 will then be transmitted (wirelessly or with a wire) to the processor 38 in the main device 12. The processor 38 in the main device 12 will convert both acceleration data sets to velocity and displacement data. The processor 38 in the main device 12 will then determine if the rescuer or first responder 11 is performing compressions and check if the displacement of the rescuer's or first responder's 11 chest compressions are above or below a given set of two thresholds. If the displacement is below both thresholds, the processor 38 will activate a visual display 35 in the main device 12 indicating to the rescuer or first responder 11 to use more force. If the displacement falls between the thresholds, the processor 38 will activate the visual display 35 in the main device 12 indicating that the force used is adequate. If the displacement is greater than both thresholds, the processor 38 will activate visual display 35 in the main device 12 indicating to the rescuer or first responder to use less force.

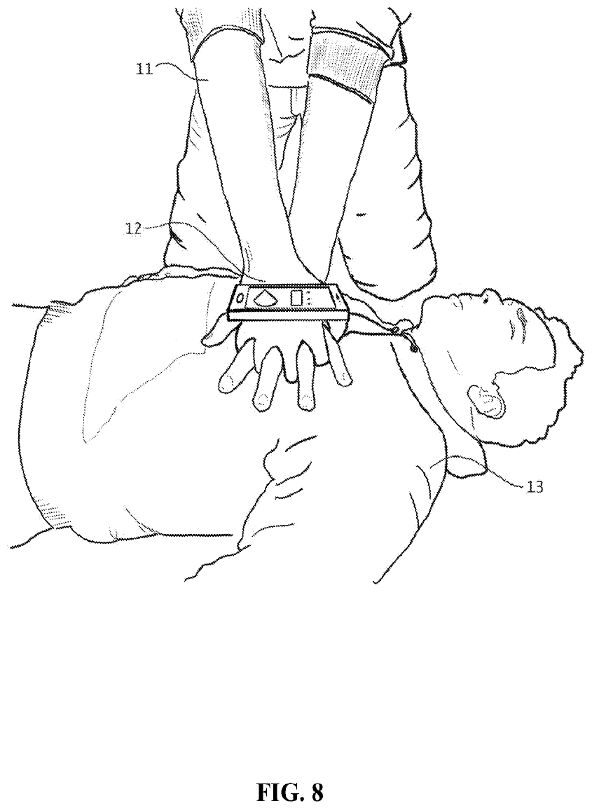

A preferred embodiment of the device will allow the non-conductive adhesives 41 and 51 and/or batteries 44 powering the main device 12 and reference device 13 to be disposable and replaceable. This disposable part of the device, as shown in FIG. 8 that depicts a second embodiment of the CPR assistive device 12 described herein that is attached to the back of the rescuer's or first responder's top hand 11 while performing CPR, could be used with the main device 12 or the reference device 13. This way after each use the partly drained batteries can be replaced with new batteries and/or the single use non-conductive adhesive will be replaced with a new non-conductive adhesive which will be ready for the device's next use. Meanwhile, all the other electronic components in the device will be made of or housed in sterilizable materials so they can be sterilized and reused. The disposable part of the device will attach to the reusable part in a non-symmetrical manner so it would be impossible to reverse the polarity of the batteries in the disposable part with respect to the electrical components in the reusable part. Alternatively, the reusable part of the device could be designed and wired so it can operate independently of battery polarity.

In an alternative embodiment, the main device 12 could also be made to give feedback indicating if the rescuer or first responder 11 is allowing the chest of the patient 14 to recoil properly. This could be done by comparing the main device's 12 accelerometer 39 data corresponding to the downstroke of the compression with the data corresponding to the upward stroke. If the rescuer or first responder 11 pushed down farther than they pulled up, then the device would indicate that there is inadequate chest recoil via the feedback interface.

In an alternative embodiment, the main device 12 could operate without the reference device 13 to reduce the cost of the device. Instead of using the reference device 13 to monitor the motion of the patient's body 14, the main device 12 assumes that the patient's body 14 is stable or use frequency analysis to differentiate chest compression movement from other movements. This would be useful in situations like a hospital setting where the patient is simply laying on a hospital bed, but it would be less desirable for situations where CPR is administered in a moving vehicle like an ambulance.

In another alternative embodiment, the thresholds discussed in Para. [0032] above, could be set by the rescuer or first responder 11 or otherwise modified before, during, or after performing CPR based on the size, age, weight, or build of the patient 14 or based on other parameters.

In another alternative embodiment, the main device 12 or the reference device 13 could be equipped with additional sensors to gather data. This data could be transmitted to the main device 13 processor 38 that would be programmed to activate or change the user feedback display 35 to provide additional feedback to the rescuer or first responder 11. The additional data gathered by the main device 12 or the reference device 13 could also be transmitted and/or saved to an external computer system or display (not shown). It could further be compared to other data to assess the patient's health.

Another alternate embodiment includes an additional non-conductive adhesive component with a separate battery, microprocessor, and wireless transmitter placed on the front or back of the rescuer's or first responder's 11 bottom hand. This could also take the form of a device placed on the victim's 14 chest. It would include accelerometers and/or pressure sensors (similar to Minami K et al., (2016), Resuscitation, 99:e11-12) to gather more data on the quality of CPR chest compressions being administered. This could improve the accuracy of the device because by providing additional data.

In another alternative embodiment, the battery 44 on the main device 12 or the reference device 13 could be made to be recharged instead of being replaced. The main device 12 and/or the reference device 13 would also include a charging port or wireless charging capabilities.

Other alternative embodiments involve the same electrical components present in the main device 12 housed in different ways that can operate independently of a reference device 13. The main device 12, according to this embodiment, is a glove worn on the rescuer's or first responder's 11 top hand with the visual display 35 on the back of the same hand. The device could also attach to the rescuer's or first responder's 11 top hand with a hook and loop fastening means such as a strap (commercially sold under the tradename Velcro® (Velcro BVBA, Deinze, BE) so the visual feedback display would be on the back of the hand. Another embodiment of the device could consist of a rigid plastic part held below the rescuer's or first responder's 11 bottom hand while in use. It would have an attached part extending around the rescuer's or first responder's 11 hands to give a visual feedback display above the rescuer's or first responder's 11 top hand. All of these alternative device housings would still take data and give feedback in the same way as discussed above.

In another alternate embodiment of the invention, the device could be modified to have two or more main devices connected to a common reference. This would allow multiple rescuers or first responders or first responders to take turns administering chest compressions, alternating when one rescuer or first responder gets fatigued.

In a further embodiment, the reference device 13 could be designed to be permanently or temporarily attached to a hospital bed, gurney or stretcher. This embodiment would not require that the device be attached directly to the patient's body. This embodiment of the claimed invention would be used in situations where the patient is injured or has a wound in the areas where the device is to be adhered.

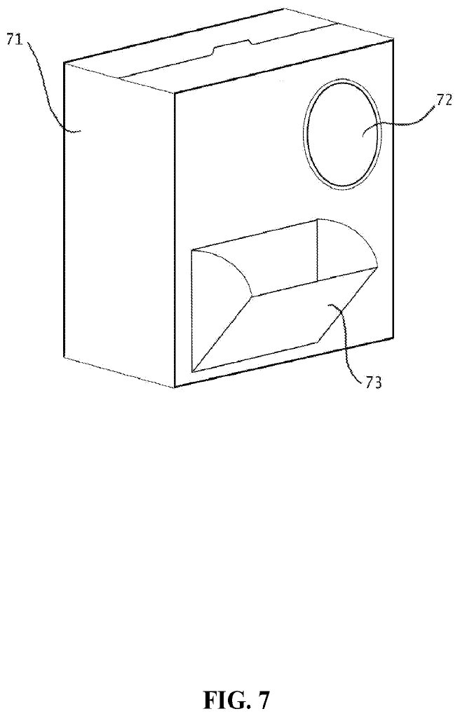

In another embodiment, the reference device 13 is designed to be attached to a hub or housing/storage container 71 used as a central location to store the other devices (and replaceable pads used to secure the device to the patient (14)) as depicted in FIG. 7. The reference device (13) 72 would be stored on the outside of the hub 71 and the main device 12 (not shown) or devices would be dispensed from a slot or opening 73 found in the wall of the hub or housing/storage container 71 allowing for the main device 12 (not shown) or devices and reference device 72 or devices to be stored in a compact container so they can be readily available for use. This hub or housing/storage container 71 could also have capabilities to recharge one or more main or reference devices with a wire or wirelessly. This hub or housing/storage container 71 could be included on or attached to a hospital crash cart or the inside of an ambulance. In some situations, the reference device 72 is left on the hub or housing/storage container 71 instead of attaching to the patient to make use of the invention more streamline.

In another alternative embodiment, the reference device 13 could be designed to record other biometrics or data points from the patient. This data could be sent to the main device 12 or an external device to be saved or used in other ways while assessing or monitoring a patient's health.

In yet a further embodiment, the main device 12 could be altered to attach to a rescuer's or first responder's 11 fingers or thumb to track depth of CPR chest compressions administered to infants.

In another embodiment, the main device 12 could also be designed to provide feedback to the rescuer or first responder on the frequency of chest compressions administered by measuring the frequency of the chest compressions then providing visual, auditory, or tactile feedback to the rescuer or first responder depending on how their frequency compares to a given target frequency. Alternatively, the main device 12 could simply act as a metronome, wherein the rescuer or first responder would match their compressions with the beat of the metronome. The metronome could be made with a speaker, making a sound for every beat administered, or with a vibration motor that would vibrate for every beat, or alternatively, a small light that blinks for every beat of the metronome.

In another alternative embodiment, the device could be programmed to suppress output when the rescuer or first responder is not performing chest compressions so as not to be distracting if the rescuer or first responder is performing rescue breaths or resting while switching off with another rescuer or first responder.

In a further embodiment, the device would only provide feedback when the rescuer or first responder deviates from the pre-programmed chest compression target. If the rescuer or first responder is performing CPR that meets the given guidelines and targets, the device does not provide any distracting information.

In an alternative embodiment, the device could be modified to be more suitable to be used in a classroom setting for training purposes. The adhesive on the main device would be removed and instead the main device would simply strap to the back of the user's hand, be held, or attached in another way so that the device may be re-used without replacing any parts. In this embodiment, the reference device 13 could be omitted because it is rendered unnecessary for most training scenarios.

In another alternative embodiment, the device could be made to sync with an automated external defibrillator (AED)(ZOLL® Medical Corporation, Chelsmford, Mass.) that is being used on the same patient. The AED, main device 12, and reference device 13 would be designed to transmit data back and forth. The AED pads (AED Brandse, Kennesaw, Ga.) that adhere to the patient's chest could also act as reference accelerometers.

In another alternative embodiment, the processor 38 on the main device 12 could be programmed to filter the accelerometer 39 data removing the component of the motion that is not directed into the patient's 14 chest. Data from a gyroscope such as the ADXRS290 gyroscope (Analog Devices, Inc., Norwood, Mass.) could be used in this filtering operation to better determine the angle of motion.

In a different embodiment, the device could be made with a visual feedback interface that is designed to be easily understood by colorblind rescuers or first responders or first responders by avoiding using combinations red, green, and yellow together in the same interface. Instead, it could use blue and orange or other sets of colors with high value difference.

In a particular embodiment, either the main device 12 or the reference device 13 could be designed to include a temperature sensor (TE Connectivity®, Tyco International Services GmbH, Schaffhausen, CH) to analyze, save, or transmit body temperature data.

In certain embodiments, the main device 12 could be designed to include an additional display indicating the time that has elapsed since the user began performing CPR helping the rescuer or first responder know if they are approaching or have exceeded a given CPR time limit.

In alternative embodiments, the device could be designed to display data indicating a history of chest compressions in addition to the real time depth data making it easier for the rescuer or first responder to read and understand than real time feedback that is rapidly changing.

In other embodiments, the main device's 12 user feedback could be paired with another device gathering and/or processing chest compression or other data which could be displayed on the back of the rescuer's or first responder's hand.

In other examples, the user interface display 35 on the main device 12 can be designed to give real time feedback about the depth of the compressions throughout the entirety of each stroke. This could be displayed as a continuous depth meter as shown in FIG. 2. The meter would indicate the point when the chest has been fully compressed, the point when the chest has fully recoiled, and a continuum of points between these positions.

Another preferred embodiment of the hardware device used in this invention is the Google™ Pixel 3a® (Google™ LLC, Mountain View, Calif.) as set discussed at https://store.aooale.com/us/product/pixel_3a_specs downloaded from the internet on Jun. 3, 2020. Other smartphones, smart watches, wearable devices, or other electronic devices with these components may still be used just as effectively.

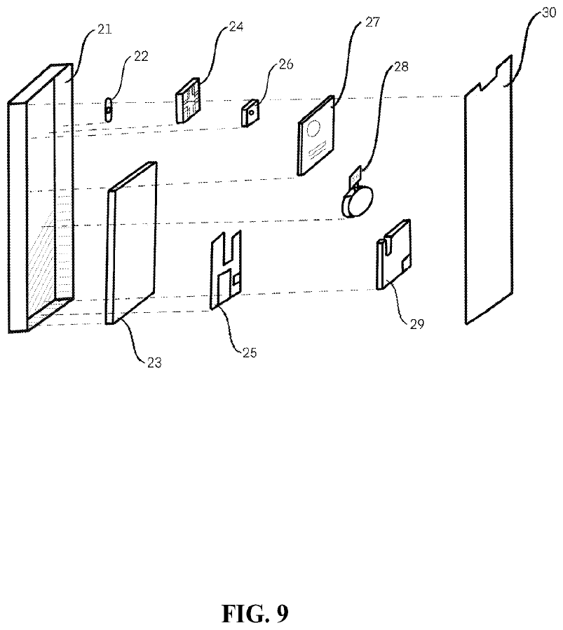

In a preferred embodiment, the invention of FIG. 9, depicts the hardware components of the device including a housing 21 containing electronics like an on switch 22, a battery 23, one or more movement or distance sensor, such as an accelerometer 24, one or more speakers 25 or a gyroscope 26 such as the ADXRS290 gyroscope (Analog Devices, Inc., Norwood, Mass.), a processor 27, one or more vibration motors 28, and one or more internal memory units 29. The device also includes a visual display 30 which is attached to the housing and can provide visual instructions or CPR feedback. Preferably, an accelerometer 24, such as the one included in Google™ Pixel 3a® (Google™ LLC, Mountain View, Calif.) and a gyroscope 26 also found in the Google™ Pixel 3a® (Google™ LLC, Mountain View, Calif.), is used.

FIG. 9 also depicts the movement sensors in the housing 21 that also contains one or more memory units 29 such as a 64 gigabyte drive memory unit included in Google™ Pixel 3a® (Google™ LLC, Mountain View, Calif.). The housing 21 may also contain one or more processors 27, such as a Qualcomm® Snapdragon™ 670 (Qualcomm Technologies, Inc., San Diego, Calif.) included in the Google™ Pixel 3a® (Google™ LLC, Mountain View, Calif.).

The same housing depicted in FIG. 9 that holds the sensors, memory, and processing hardware also houses one or more actuators for conveying information, feedback, and/or instructions to the user. In a preferred embodiment, the housing 20 would hold one or more speaker 25, such as stereo speakers, as included in the Google™ Pixel 3a® (Google™ LLC, Mountain View, Calif.) as well as a visual display 30, such as a 5.6 inch screen which is included in the Google™ Pixel 3a® (Google™ LLC, Mountain View, Calif.). This embodiment may further comprise haptic feedback capabilities, such as the vibration motors 28 included in the Google™ Pixel 3a (Google™ LLC, Mountain View, Calif.).

This embodiment of the invention shown in FIG. 9 is powered by a battery 23 that is also contained in the same housing 21. Preferably, a 3000 milliamp hour battery (Duracell®, Inc., Betherl, Conn.) would be used, as included in the Google™ Pixel 3a® (Google® LLC, Mountain View, Calif.). The housing 21 also includes a button 22 to turn the device on and off. Preferably, a power button similar to the power button found in the Google™ Pixel 3a® (Google™ LLC, Mountain View, Calif.). would be used. Electrical connections would also be included to interface all the described components to the battery and processor.

The device's processor 38 may be programmed to use the accelerometer 39 to take measurements of the patient's 14 chest movement and/or the rescuer's or first responder's 11 hand movements as a rescuer or first responder 11 is performing CPR; transferring the data to and stored in the memory unit 29, then processed by the processor 38 using an algorithm to convert the accelerometer data into compression depth data. This depth data will also be transferred to and stored on the memory unit 29.

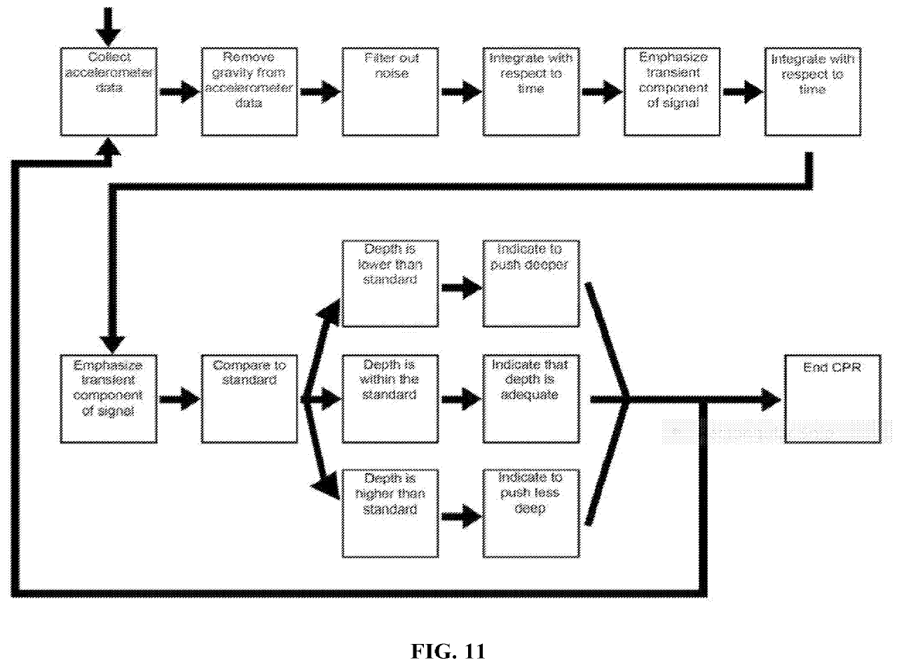

The algorithm of FIG. 11 shows that the conversion accelerometer data to compression depth data requires first subtracting the component of the signal that corresponds to the gravitational field felt by the device. The algorithm would then take the remaining component and filter out any noise using a high-pass filter (Maxxcom, Inc., Fair Oaks, Calif.) or any other method known by those skilled in the art. The signal would then be integrated with respect to time twice. Transient components of the signal may need to be emphasized between integrations and or after both integrations. This will yield a result corresponding to chest compression depth. There are obviously a great many alternative algorithms that could be used to get to the same result. The described algorithm is preferred.

The processor 38 will then compare the compression depth data to a relevant standard on compression depth such as the standard set by the American Heart Association® (American Heart Association®, Inc., Dallas, Tex.). If the rescuer's or first responder's 11 compression depth is lower than the standard, the device will use one or more of the actuators to indicate to the rescuer or first responder 11 that they need to push deeper into the chest. If the rescuer's or first responder's 11 compression depth meets the standard, the device will use one or more of the actuators to indicate to the rescuer or first responder 11 that they reached the appropriate compression depth. If the rescuer's or first responder's 11 compression depth is greater than the standard, the device will use one or more of the actuators to indicate to the rescuer or first responder 11 that they should push less deep into the chest. In a preferred embodiment, when the device is indicating chest compression depth recommendations to the rescuer or first responder 11, it would display this recommendation on the visual display unit 35 in the form of a diagram and/or text. It could also use the speakers 25 to give auditory instructions on compression depth. Furthermore, haptics could be used to briefly activate the vibration motors 28 when the optimal chest compression depth is reached. Any information that is output auditorily, visually, or using haptics could also be stored in the memory unit 29 to be reviewed later by the rescuer or first responder or a medical professional.

The device may further comprise additional sensors such as a camera, magnetometer, button, touch screen or other sensors. The device may also use the accelerometer 39 and/or these additional sensors to measure additional CPR-related parameters such as chest compression rate, chest recoil, elapsed time, and more. These parameters may be stored. Feedback on these parameters may be given to the rescuer or first responder as well. These parameters may also be transmitted to another nearby device or a remote location where the information can be stored and/or reviewed by a medical professional, trained professional, or an additional algorithm. The professional or algorithm may also send instructions or information back to the rescuer or first responder as a response to the received data.

In the preferred embodiment, the device giving CPR feedback would also comprise wireless connectivity capabilities including sending and receiving data and other files such as found in the Google™ Pixel 3a® (Google™ LLC, Mountain View, Calif.) or similar devices known by those skilled in the art. This device could be held by the rescuer or first responder, attached to their hand or wrist or otherwise positioned to move with the rescuer's or first responder's hands or the patient's chest while performing CPR. The device would use the built in accelerometer 39 and gyroscope 26 to measure the acceleration of chest compressions. This can be used to determine chest compression depth as described. Chest compression rate may also be measured using this data. This information would be displayed on a screen, such as the 5.6 inch screen employed by the Google™ Pixel 3a® (Google™ LLC, Mountain View, Calif.), for the rescuer or first responder to see. Auditory and tactile feedback could also be given using the device's built in hardware. This embodiment is depicted in FIG. 8.

A further embodiment of this invention may additionally be comprised to communicate or display instructional information or directions that are relevant for performing CPR. These instructions would be displayed before CPR feedback is given and may be comprised of text and/or diagrams. The instructions may include, but are not limited to, the following steps: 1). checking if the patient is responsive; 2) checking if the patient is breathing; 3). Ensuring that the patient is on a stable, hard surface; 4). positioning the feedback device in a certain way; 5). positioning the rescuer's or first responder's hands in a certain way; and 6). commencing compressions of the patient's chest. The rescuer or first responder could have the option to skip instructions so they can read some, all, or none of the instructions depending on their training level and familiarity with CPR and related procedures. One way in which this could be achieved would be to have a setting for professional rescuers or first responders or first responders, who may not need as much guidance, and another for non-professional rescuers or first responders or first responders, who may require more guidance. The setting for professional rescuers or first responders or first responders could also make the system display additional CPR relating information and/or feedback such as chest recoil or elapsed time. The feedback and display could further be customized using other settings.

Another embodiment of this invention allows the rescuer or first responder to select the approximate age range of the patient on which they are performing CPR. These ranges may include infant (0 to 1 years old), child (1 to 8 years old), and adult (8+ years old). The rescuer or first responder will select the appropriate range before beginning CPR. Once CPR has commenced, the data collected will be compared to standards specific to the age group selected. The feedback provided will therefore be correct for patients of any age group.

An embodiment of this invention may also be capable of automatically detecting when compressions begin. Once compressions are commenced, the device may change from giving instructions to providing CPR feedback without additional rescuer or first responder input. This could be done by using data corresponding to the device's position or movement and looking for key features of the position or movement which are distinct to chest compressions.

An embodiment of this invention may also count chest compressions and/or record time. This information would be displayed for the rescuer or first responder so that the rescuer or first responder is aware when to perform rescue breaths, administer medication, apply a defibrillator shock, or switch the responsibility of performing chest compressions with another rescuer or first responder.

An further embodiment of this invention may also provide a visual, tactile, and/or auditory metronome to help the user perform chest compressions at a given rate determined by CPR standards. The visual metronome may be displayed as an oscillating symbol with a stationary symbol along the route of oscillation where the symbols meet at a given frequency.

An embodiment of this invention may also be able to determine chest recoil. When chest recoil is determined to be inadequate, the feedback system would trigger, informing the user they need to ensure proper chest recoil between compressions.

An embodiment of this invention may also save and/or upload the collected data for future reference. Chest compression parameters can be saved and/or uploaded to future review as well. This can be saved in the form of a csv file or other type of file. These files can be sent to a cloud storage system or another device using Bluetooth® (Bluetooth® Sig, Inc., Kirkland, Wash.) or other wireless technology. This can be used to look back and access CPR performance or do code reviews from the device that was used during CPR or other devices.

In yet another embodiment of instant invention, data or chest compression parameters to a separate device is transmitted using any known data transmission devices, such as smart glasses, or other devices capable of transmitting auditory and/or visual feedback. This device may be operated by another rescuer or first responder who can use the information and verbally coach the rescuer or first responder doing chest compressions. This method of human coaching may be preferable for some rescuer or first responders or first responders.

Further embodiments of this invention may also collect data from sensors that are not housed in the device that contains the feedback system. These sensors may include, but are not limited to, a cardiac monitor, an electrocardiogram, a camera, a blood flow sensor, and/or other sensors known to those skilled in the art. These sensors could be housed separately and transmit the data to the main device 12 via a wire or wireless connection or transmit the device to a cloud storage system or other device for future review.

An embodiment of this invention may also be capable of alerting local authorities and professional medical responders and/or transmit location data automatically and/or when prompted.

An embodiment of this invention may be capable of calling the ambulance directly from the device without exiting the relevant software. An extension of this may include transmitting location data to an ambulance or ambulance dispatch service. An additional feature may comprise an ambulance sending updates regarding estimated time of arrival on scene.

Another embodiment of this invention may also require the user to pay for the app to use it or after a free trial period is over.

### Method

The described device is meant to be used by both professional and non-professional rescuer or first responders. Steps for use may include some or all of the following steps in any order:

1). recognizing a patient may need CPR and activating the device;

2). viewing the device's instructions;

3). beginning CPR and chest compressions; and

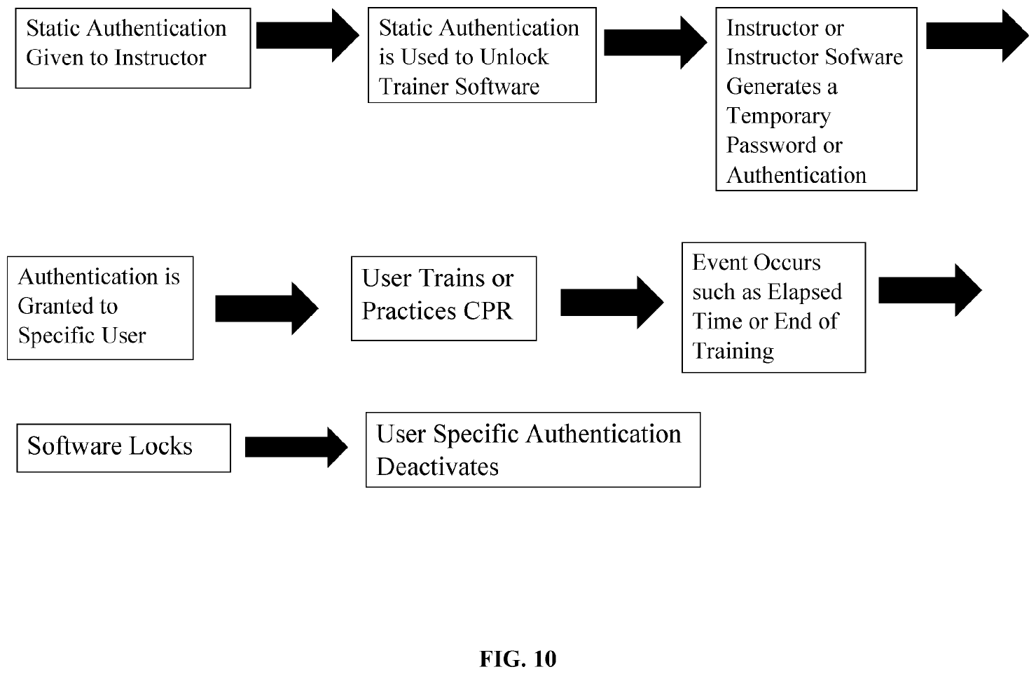

4). using feedback and queues from device to adjust chest compression depth rate, chest recoil or other parameters. An implementation of the method is illustrated in the flow chart of FIG. 10. The mentioned device's instructions may include checking if the patient is responsive, checking if the patient is breathing, and positioning the device properly.

Additionally, steps may be added to transmit CPR related data while performing CPR and/or transmitting CPR-related data after performing CPR. While one rescuer is using the device as described, another rescuer or first responder may use another device to receive transmitted data and provide verbal coaching to the first rescuer or first responder who is performing chest compressions. The data can also be transmitted to medical professionals, emergency services dispatchers, or cloud storage units.

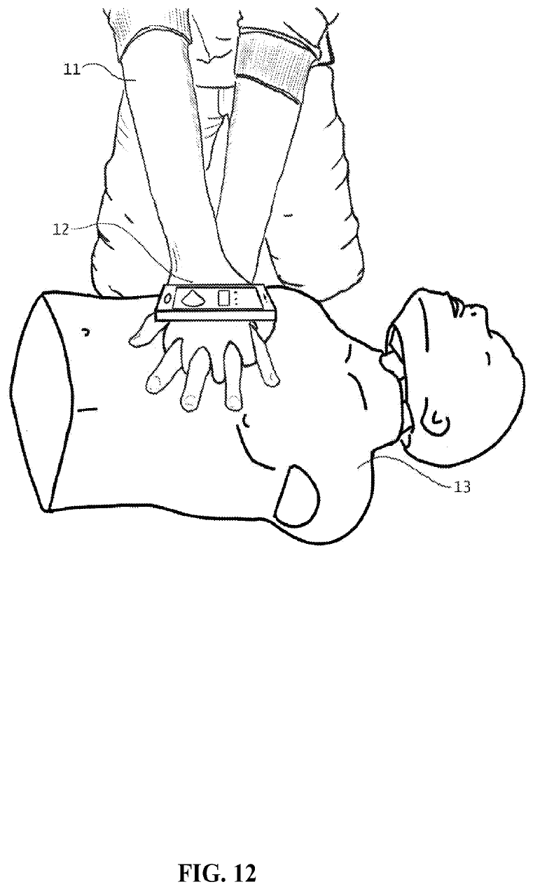

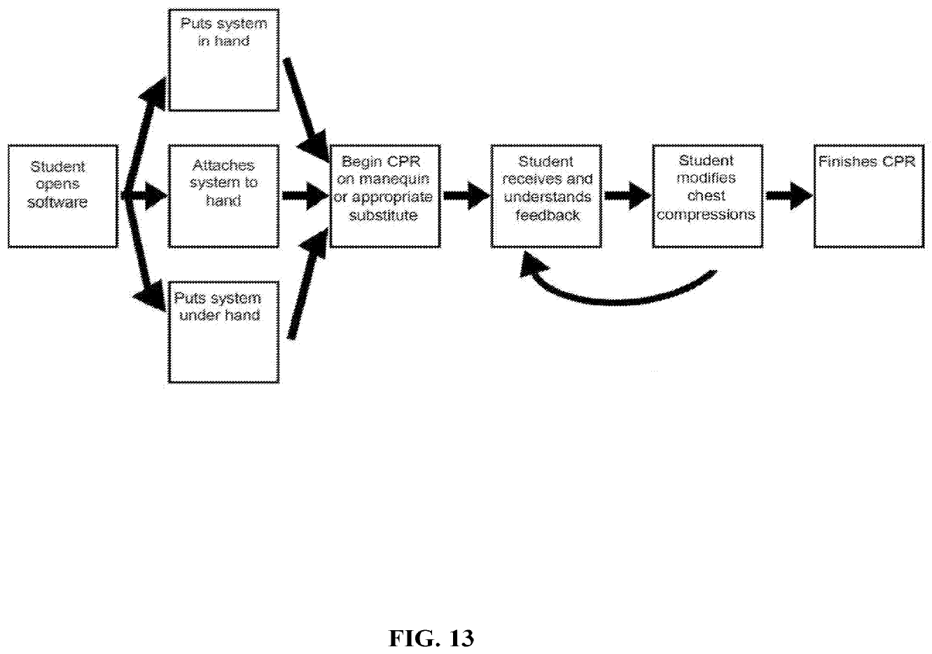

The described device can also be used in the following way to aid a student in learning or practicing CPR. The student holds the described device or the sensing part of the device, attaches it to their hand, attaches it to their wrist or places it under their hand in contact with the CPR mannequin's chest at the compression site. When the student begins compressions, the device gives feedback to the student and/or the instructor on chest compression depth, chest compression rate, and/or other CPR parameters. The student then views, listens to, or feels the feedback. The student can then adjust their chest compression rate, depth, or other CPR related actions based on the feedback and as needed. FIG. 12 depicts usage of a preferred embodiment of the invention. FIG. 13 shows an implementation of steps that may be involved. Additionally, the student may view instructional content on the same device or a different device, such as videos, text, and/or images, and/or answer one or more quiz questions as a part of this method of use.

The CPR data collected by the sensors while the student is practicing CPR or CPR parameters calculated based on the collected data and other data may be stored on the device in a memory unit. This would allow the data or parameters to be reviewed during or after the student finishes practicing CPR. Additional parameters could also be calculated retrospectively such as the percent of compressions that reached a proper depth. This could be viewed by the student or the student's instructor to assess the student's performance and/or decide if the student needs additional training or practice. The parameters could also be compared to other thresholds such as a threshold corresponding to average performance, expected performance or someone else's performance so that the student can better understand their own performance.

Instead of using a device with an accelerometer as the movement sensor, a device with a camera, such are the camera incorporated into the Dell EMC® Inspiron® laptop (Dell, Inc., Round Rock, Tex.), is employed. The camera can record video of the student practicing CPR. The student's hands or the mannequin's chest can be tagged either virtually or physically for object tracking. Physical tags may include a marking, sticker, glove or wristband. Image processing algorithms can use this video data to determine chest compression depth, chest compression rate, chest recoil or other CPR parameters.

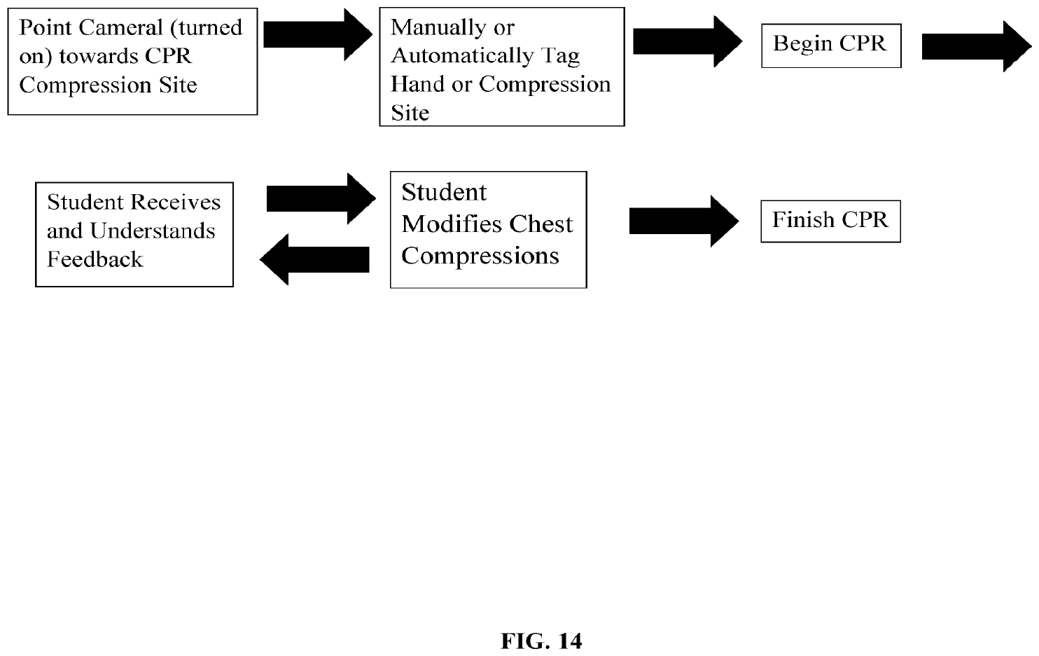

One method to determine chest compression depth involves putting an object or marking of known dimensions in the camera's frame as a reference distance to calibrate the measurement. The device will be programmed to recognize the tag and track the motion of the tag over time. This can be compared to the reference distance to determine the distance the tag has traveled, indicating chest compression depth. Other methods of determining compression depth may also be used. Other parameters, such as rate and chest recoil, may not need this reference object for accuracy. An implementation of this method is depicted in FIG. 14.

This CPR training method may be used with any CPR mannequin, but it is additionally useful when using a mannequin that does not give feedback, such as a low-cost cardboard or inflatable mannequin. This method may also be performed on a pillow, couch cushion, other compliant object, or even in mid-air.

This CPR training method may also be administered by an instructor who is not physically present, but visually communicating with the student through an audio or voice chat such as Skype (Skype®, Dublin, IE). It also may be administered automatically through a smartphone or a computer application. This enables a student to be trained in CPR remotely, and from any location, such as their own home, for added convenience. In this situation, the CPR parameters, signals, and/or feedback may also be wirelessly transmitted to the instructor.

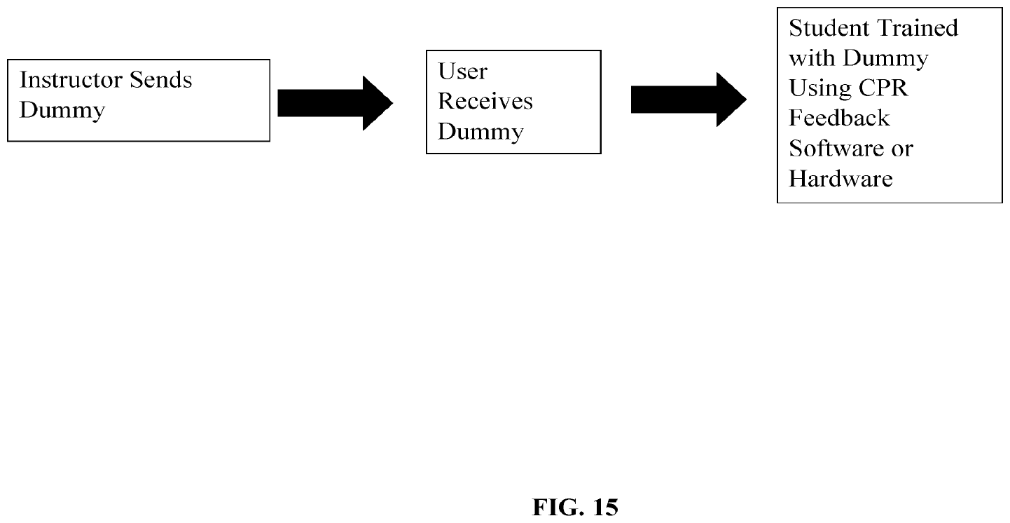

Another step which may be added to this method is for a CPR training company, other company, or individual to send a low cost CPR mannequin to the student, or the student's employer, or nearby location through a mail service or other delivery method. This further adds convenience for the student. If this mannequin does not give CPR feedback or only gives partial feedback then feedback can be provided using the device and methods described above and/or further steps described below. Instructions for using the mannequin and/or CPR feedback can be sent with the mannequin or can be sent electronically or can be given directly by the instructor. An implementation of steps that may be involved in this is shown in FIG. 15.

Chest compression signals, parameters or CPR feedback may be recorded locally or transmitted to instructors, employers or reviewers for data analysis or analysis of performance. In addition to giving feedback to the student, the data can be used to determine if the student needs further instructions, either in real time or after the student finishes performing CPR. The data can also be used to determine if the student needs additional training or practice or if the student should be issued a CPR training certificate.

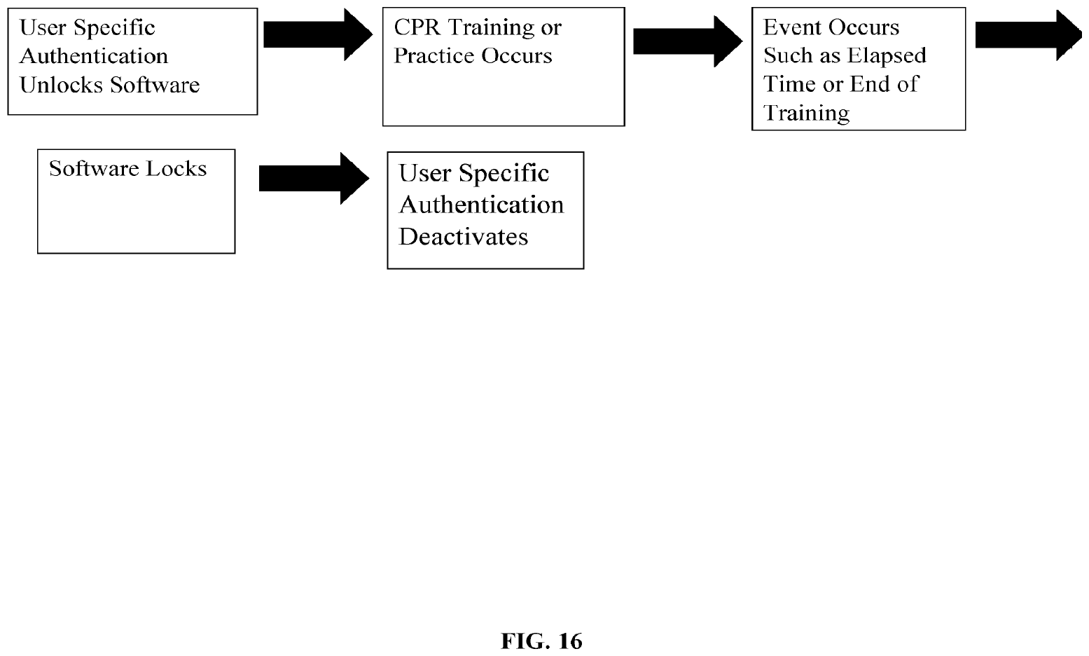

When the device with sensors is used to provide CPR feedback, a passcode or other authentication system may be used to ensure that the feedback enabling software on the device is only used for training or used in other approved situations. One implementation of this would be to provide a pass code to the student so that the student can unlock the software before the student uses the software. The program could also be set to close out or lock again when a certain condition or conditions are met, such as a time limit, the end of a training session and/or a signal from the instructor. An graphic depiction of this process is presented in FIG. 16.

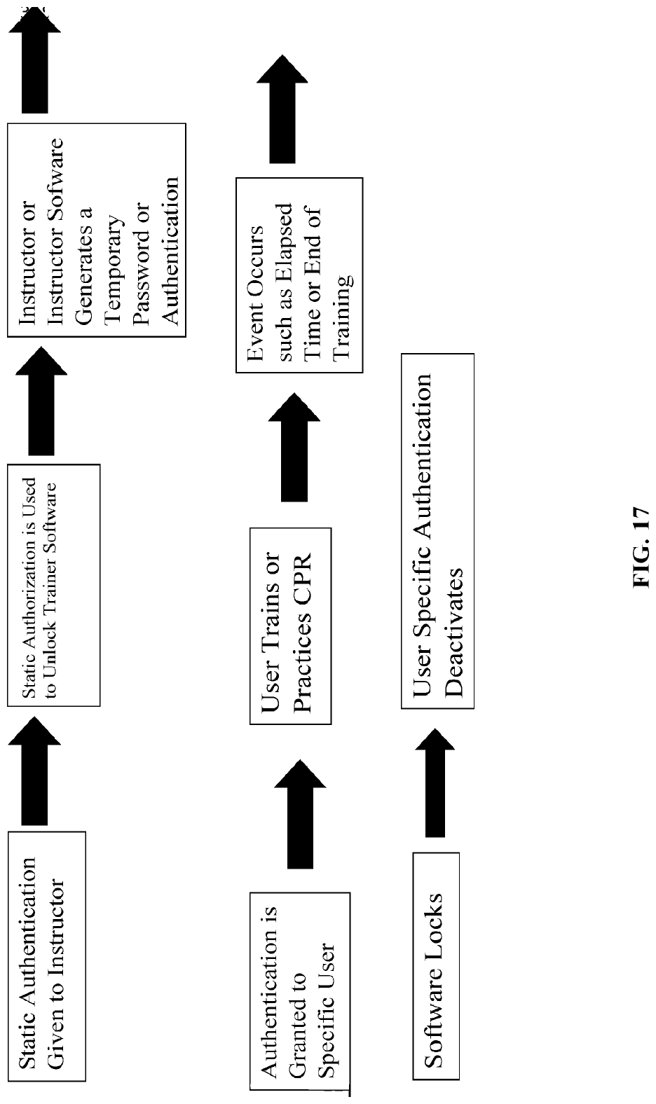

An implementation of the authentication system used to grant access to the software may involve a double authentication system. A static password or other authentication could be selectively granted to certain CPR instructors. This static password allows for the instructor to unlock or sign into a CPR training software package. The instructor generates a temporary password that the instructor then discloses to the student. This temporary password allows the student to use the CPR feedback software on a device in unapproved situations by re-using the password or granting themselves access in other ways. An graphic depiction of this process is presented in FIG. 17.

Another method to ensure a student uses the training version of the CPR feedback software for training purposes only and not in a real world emergency situation is to impose a waiting period between the time that the student uses the software for training purposes and the time when the student received feedback about the recorded CPR parameters. If the waiting period is sufficiently long, the student would not be able to use the feedback software in an emergency situation because by the time the student is able to receive feedback would be too late. The waiting period could be occupied by training videos, quizzes and other content.

An additional step of collecting payment for use of the device may be added. The software would record use and/or the number of uses of an authentication key. This data would then be used to accurately bill either the student or instructor for use of the service. This billing process may be automated.

### Definitions

For convenience, certain terms employed in the specification, examples and appended claims are collected here. These definitions should be read in light of the disclosure and understood as by a person of ordinary skill in the art.

As used herein, the term “actuator,” refers to a component of a machine that is responsible for moving and controlling a mechanism or system.

As used herein, the term “administering,” “administer” or “administration,” refer to the act of dispensing or applying.

As used herein, the term “dummy” or “mannequin” refers to an object that is compressible by human force.

As used herein, the term “first responder” or “rescuer,” refers to any person providing care to a patient, including but not limited to non-professional rescuer or first responders or first responders, right fighters, emergency medicine technicians, police officers, or nurses.

As used herein, the term “haptics,” refers to the use of technology that stimulates the senses of touch and motion, especially to reproduce in remote operation or computer simulation the sensations that would be felt by a user interacting directly with physical objects.

As used herein, the term “measure” or “measuring,” refers to ascertain the size, amount, or degree of (something) by using an instrument or device which may include one or more sensors, and/or computational ability for performing operations on sensor data.

As used herein, the term “parameter(s),” refers to a numerical or other measurable factor forming one of a set that defines a system or sets the conditions of its operation.

As used herein, the term “selected,” refers to carefully choose as being the best or most suitable.

As used herein, the terms “comprises.” “comprising.” “includes.” “including.” “has “having or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, ‘or’ refers to an inclusive or and not to an exclusive or. For example, a condition A or B is satisfied by any one of the following: A is true (or present) and B is false (or not present), A is false (or not present) and B is true (or present), and both A and B are true (or present). Also, use of the “a” or “an” are employed to describe elements and components of the invention. This is done merely for convenience and to give a general sense of the invention. This description should be read to include one or at least one and the singular also includes the plural unless it is obvious that it is meant otherwise. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. In the following description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment in various places throughout this specification are not necessarily all referring to the same embodiment. Furthermore, the particular features, structures, or characteristics may be combined in any Suitable manner in one or more embodiments.

The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B, and C components.

### EQUIVALENTS

The full scope of the invention should be determined by reference to the claims, along with their full scope of equivalents, and the specification, along with such variations.

Unless otherwise indicated, all numbers expressed quantities of ingredients, reaction conditions, and so forth use in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

We claim:

1. A cardiopulmonary resuscitation monitoring device used by a first responder capable of measuring selected parameters during the administration of cardiopulmonary resuscitation to a patient in need thereof.

2. The cardiopulmonary resuscitation monitoring device according to claim 1, wherein said device provides said measurements in real time.

3. The cardiopulmonary resuscitation monitoring device according to claim 2, wherein said parameters are selected from the group consisting of the depth of chest compressions applied to the patient and the frequency of said chest compressions administered to the patient.

4. The cardiopulmonary resuscitation monitoring device according to claim 1, wherein said device may be used in a moving ambulance.

5. The cardiopulmonary resuscitation monitoring device according to claim 1, wherein said device may be used by a first responder wearing light gloves.

6. The cardiopulmonary resuscitation monitoring device according to claim 1, wherein said device comprises programming that evaluates said parameters, identifies problems with the and provides guidance to a first responder as to how to correct said problems.

7. The cardiopulmonary resuscitation monitoring device according to claim 1, comprising one or more sensors.

8. The cardiopulmonary resuscitation monitoring device according to claim 7, wherein said sensors detect said parameters.

9. The cardiopulmonary resuscitation monitoring device according to claim 8, wherein said parameters are selected from the group consisting of the depth of compressions applied to the patient and the frequency of compressions administered to the patient.

10. The cardiopulmonary resuscitation monitoring device according to claim 7, further comprising means in which to secure said device to a hand of said first responder.

11. The cardiopulmonary resuscitation monitoring device according to claim 10, wherein said one or more sensor is attached to said device and positioned on a first responder's lower hand, in a first responder's hand, or on the back of a first responder's hand.

12. The cardiopulmonary resuscitation monitoring device according to claim 11, further comprising a feedback data transmission system.

13. The cardiopulmonary resuscitation monitoring device according to claim 12, wherein said feedback data transmission system transmits visual, audio and tactile information about the patient on which cardiopulmonary resuscitation is being performed.

14. The cardiopulmonary resuscitation monitoring device according to claim 13, further comprising a visual feedback interface capable of displaying said visual, audio and tactile information.

15. The cardiopulmonary resuscitation monitoring device according to claim 14, wherein said visual feedback interface is incorporated into the means in which to secure said device to the hand of said first responder and is positioned so as to be visible to the first responder when said device is worn by said first responder during the administration of cardiopulmonary resuscitation on a patient.

16. The cardiopulmonary resuscitation monitoring device according to claim 14, further comprising a reference device affixed to the patient in need thereof cardiopulmonary resuscitation.

17. The cardiopulmonary resuscitation monitoring device according to claim 16, wherein said reference device is positioned on a patient in need of cardiopulmonary resuscitation.

18. The cardiopulmonary resuscitation monitoring device according to claim 17, wherein said reference device is positioned on the neck or the distal portion of the ribs or any stable part of the patient.

19. The cardiopulmonary resuscitation monitoring device according to claim 16, further comprising one or more accelerometers incorporated into the means in which to secure said device to the hand of said first responder wherein said one or more accelerometers measures the depth of displacement of a first responder's hands while performing chest compressions on the patient and the frequency of the chest compressions administered to the patient subject to the cardiopulmonary resuscitation being performed.

20. The cardiopulmonary resuscitation monitoring device according to claim 16, further comprising one or more accelerometers incorporated into said reference device positioned on the patient subject to the cardiopulmonary resuscitation being performed.

21. The cardiopulmonary resuscitation monitoring device according to claim 20, wherein said one or more accelerometers measure changes in the position of the patient subject to the cardiopulmonary resuscitation being performed.

22. The cardiopulmonary resuscitation monitoring device according to claim 19, further comprising one or more microprocessors.

23. The cardiopulmonary resuscitation monitoring device according to claim 22, wherein said one or more microprocessors is incorporated into the means in which to secure said device to the hand of said first responder and incorporated into the reference device affixed to the patient.

24. The cardiopulmonary resuscitation monitoring device according to claim 22, wherein said microprocessor is programmed to differentiate movements of the patient from movement of the first responder's hand.

25. The cardiopulmonary resuscitation monitoring device according to claim 22, wherein said reference device positioned on the patient records movement of the patient.

26. The cardiopulmonary resuscitation monitoring device according to claim 25, wherein said patient movement data is collected by the microprocessor incorporated into the reference device positioned on the patient.

27. The cardiopulmonary resuscitation monitoring device according to claim 25, wherein said patient movement data is transmitted from the microprocessor incorporated into the reference device positioned on the patient to the microprocessor incorporated into the means in which to secure said device to the hand of said first responder wirelessly.

28. The cardiopulmonary resuscitation monitoring device according to claim 25, further comprising a wire in which to transmit said patient movement data from the microprocessor incorporated into the reference device positioned on the patient to the microprocessor incorporated into the means in which to secure said device to the hand of said first responder.

29. The cardiopulmonary resuscitation monitoring device according to claim 1, wherein said device is designed to be worn on either the right or left hand of the first responder.

30. The cardiopulmonary resuscitation monitoring device according to claim 1, wherein said device is designed to be worn by either a male or female or both sexes first responder.

31. The cardiopulmonary resuscitation monitoring device according to claim 14, wherein said visual feedback interface is capable of indicating whether the depth of a chest compression was adequate, too shallow or too deep.

32. The cardiopulmonary resuscitation monitoring device according to claim 10, further comprising frequency indicator.

33. The cardiopulmonary resuscitation monitoring device according to claim 32, wherein said frequency indicator comprises a vibration motor, blinking diode and/or speaker capable of pulsing when a desired chest compression pace is achieved.

34. The cardiopulmonary resuscitation monitoring device according to claim 33, further comprising a power source.

35. The cardiopulmonary resuscitation monitoring device according to claim 34, wherein said power source is a battery.

36. The cardiopulmonary resuscitation monitoring device according to claim 34, further comprising means in which to secure said power source to said device.

37. The cardiopulmonary resuscitation monitoring device according to claim 36, further comprising snaps that connect the feedback data transmission system, the one or more accelerometers and the microprocessor and/or other electronic components requiring power to the power source.

38. The cardiopulmonary resuscitation monitoring device according to claim 37, further comprising conductive strips connected to said power source wherein said conductive strips carry current to said snaps.

39. The cardiopulmonary resuscitation monitoring device according to claim 36, further comprising a housing having a bottom wall, four vertical walls and a cover.

40. The cardiopulmonary resuscitation monitoring device according to claim 39, wherein said housing is a component of said means in which to secure said device to the hand of said first responder.

41. The cardiopulmonary resuscitation monitoring device according to claim 40, wherein said housing contains the one or more sensors, the feedback data transmission system, the one or more accelerometers, the microprocessor, the frequency indicator, the power source and optionally an end of the wire used to transmit patient movement data from the reference device attached to the patient.

42. The cardiopulmonary resuscitation monitoring device according to claim 41, wherein said housing comprises a top cover.

43. The cardiopulmonary resuscitation monitoring device according to claim 42, wherein said visual feedback interface is embedded into said top cover of said housing.

44. The cardiopulmonary resuscitation monitoring device according to claim 43, wherein said vibration motor, diode or speaker is embedded into said top cover of said housing.

45. The cardiopulmonary resuscitation monitoring device according to claim 38, wherein said means in which to secure said device to the hand of said first responder is an adhesive.

46. The cardiopulmonary resuscitation monitoring device according to claim 45, wherein said adhesive does not conduct electricity.

47. The cardiopulmonary resuscitation monitoring device according to claim 45, wherein said adhesive is affixed to the bottom wall of said housing.

48. The cardiopulmonary resuscitation monitoring device according to claim 39, said housing is manufactured from a polymer.

49. The cardiopulmonary resuscitation monitoring device according to claim 48, wherein said polymer is semiflexible.

50. The cardiopulmonary resuscitation monitoring device according to claim 49, wherein said power source and adhesive means are disposable and replaceable.

51. The cardiopulmonary resuscitation monitoring device according to claim 50, wherein said device is sterilizable and reusable.

52. The cardiopulmonary resuscitation monitoring device according to claim 51, wherein said disposable power source attaches to the conductive strips in a non-symmetrical manner; and further wherein said non-symmetrical attachment prevents the user from inserting the power source into said device in a manner that reverses the polarity of the power source damaging said device.

53. The cardiopulmonary resuscitation monitoring device according to claim 34, further comprising means in which said device is capable of operating independently of the polarity of the power source.

54. The cardiopulmonary resuscitation monitoring device according to claim 1, wherein said device can indicate to the user if said user is allowing the chest of the patient upon whom cardiopulmonary resuscitation is being performed thereon to recoil properly.

55. The cardiopulmonary resuscitation monitoring device according to claim 54, further comprising programming that compares data from the accelerometer embedded in the housing of the claimed device.

56. The cardiopulmonary resuscitation monitoring device according to claim 55, wherein said compared data consists of the depth of the downstroke depression and the height of the upward recoil achieved during cardiopulmonary resuscitation performed on the patient.

57. The cardiopulmonary resuscitation monitoring device according to claim 56, wherein said device would advise the first responder of inadequate chest recoil if the downstroke depression is at a greater depth than the height of the upward recoil.

58. The cardiopulmonary resuscitation monitoring device according to claim 57, wherein said visual feedback interface would indicate that that the patient's chest is not recoiling properly.

59. The cardiopulmonary resuscitation monitoring device according to claim 1, further comprising means in which to differentiate chest compression movements from movements of the patient, first responders and/or other movements in the surroundings of the patient.

60. The cardiopulmonary resuscitation monitoring device according to claim 59, wherein said device uses frequency analysis to differentiate between said movements.

61. The cardiopulmonary resuscitation monitoring device according to claim 3, further comprising programming that utilizes physical characteristics of the patient upon whom cardiopulmonary resuscitation is to be performed thereon to optimize said parameters.

62. The cardiopulmonary resuscitation monitoring device according to claim 61, wherein said physical characteristics are entered into said device prior to administering cardiopulmonary resuscitation to the patient.

63. The cardiopulmonary resuscitation monitoring device according to claim 62, wherein said physical characteristics are selected from the group consisting of physical size, age, weight, build of the patient and combinations thereof.

64. The cardiopulmonary resuscitation monitoring device according to claim 1, further comprising additional sensors that collect data which is transmitted to the microprocessor contained in said housing wherein said data from said additional sensors is transmitted to and/or saved to an external computer system and/or display.

65. The cardiopulmonary resuscitation monitoring device according to claim 64, wherein said transmitted data is utilized by a third party to assess the health of the patient upon whom cardiopulmonary resuscitation is being administered.

66. The cardiopulmonary resuscitation monitoring device according to claim 65, wherein the device receives data from an independent entity or a third party and said device reacts to said data.

67. The cardiopulmonary resuscitation monitoring device according to claim 66, wherein said independent entity or third party is a primary care physician and/or a medical specialist treating the patient upon whom cardiopulmonary resuscitation is being administered.

68. The cardiopulmonary resuscitation monitoring device according to claim 1, further comprising a second housing containing a separate power source, wireless transmitter and microprocessor.

69. The cardiopulmonary resuscitation monitoring device according to claim 68, further comprising an adhesive means.

70. The cardiopulmonary resuscitation monitoring device according to claim 69, wherein said housing is attached to a first responder's bottom hand used during administration of cardiopulmonary resuscitation to a patient in need thereof or to the body of said patient.

71. The cardiopulmonary resuscitation monitoring device according to claim 70, wherein said second housing is attached to the chest of the patient.

72. The cardiopulmonary resuscitation monitoring device according to claim 68, further comprising one or more accelerometer and/or one or more pressure sensors wherein said one or more accelerometer and/or one or more pressure sensors measure the depth of the chest compressions and chest recoils during the administration of cardiopulmonary resuscitation.

73. The cardiopulmonary resuscitation monitoring device according to claim 34, wherein said power source is rechargeable.

74. The cardiopulmonary resuscitation monitoring device according to claim 73, further comprising a charging port.

75. The cardiopulmonary resuscitation monitoring device according to claim 74, further comprising means in which to wirelessly charge said power source.

76. The cardiopulmonary resuscitation monitoring device according to claim 14, wherein said means in which to secure said device to the hand of said first responder is a glove worn by said first responder.

77. The cardiopulmonary resuscitation monitoring device according to claim 10, wherein said visual feedback interface is embedded into the outside surface of said glove.

78. The cardiopulmonary resuscitation monitoring device according to claim 10, wherein said means in which to secure said device to the hand of said first responder comprises one or more hook and loop adhesive straps.

79. The cardiopulmonary resuscitation monitoring device according to claim 44, wherein said means in which to secure said device to the hand of said first responder comprises a handheld grip with an outwardly extending arm having the visual feedback interface at the end of said outwardly extending arm affixed thereto so that the first responder is able to observe said visual feedback interface during administration of cardiopulmonary resuscitation on the patient.

80. The cardiopulmonary resuscitation monitoring device according to claim 44, further comprising a second set of one or more sensors, feedback data transmission system, one or more accelerometers, a microprocessor, a frequency indicator, a power source and optionally an end of the wire used to transmit patient movement data from the reference device attached to the patient and/or vibration motor and/or diode and/or speaker.

81. The cardiopulmonary resuscitation monitoring device according to claim 44, further comprising a second housing having a top lid containing said second set of one or more sensors, feedback data transmission system, one or more accelerometers, a microprocessor, a frequency indicator, a power source and optionally an end of the wire used to transmit patient movement data from the reference device attached to the patient and/or vibration motor and/or diode and/or speaker; wherein and a visual feedback interface is embedded into the top lid of said housing; and wherein said second housing allows for the monitoring of more than one first responder administering cardiopulmonary resuscitation to the patient in need thereof.

82. The cardiopulmonary resuscitation monitoring device according to claim 12, wherein said feedback data transmission system is permanently or temporarily affixed to an object in the vicinity of the patient upon whom cardiopulmonary resuscitation is being performed.

83. The cardiopulmonary resuscitation monitoring device according to claim 82, wherein said object in the vicinity of the patient upon whom cardiopulmonary resuscitation is being performed is selected from the group consisting of a hospital bed, gurney, stretcher, hospital crash cart and the interior of an ambulance or other transportation device used by a first responder.

84. The cardiopulmonary resuscitation monitoring device according to claim 12, wherein said feedback data transmission system is permanently or temporarily or detachably affixed to the hub or the outer-housing and/or storage container for a secondary device.

85. The cardiopulmonary resuscitation monitoring device according to claim 12, wherein said feedback data transmission system is affixed to an outer surface of said outer-housing and/or storage container for a secondary device.

86. The cardiopulmonary resuscitation monitoring device according to claim 85, wherein said housing having a top lid containing said one or more sensors, feedback data transmission system, one or more accelerometers, a microprocessor, a frequency indicator, a power source and optionally an end of the wire used to transmit patient movement data from the reference device attached to the patient and/or vibration motor and/or diode and/or speaker is situated within the hub or outer-housing or storage container for a secondary device.

87. The cardiopulmonary resuscitation monitoring device according to claim 85, wherein said device is contained within a hub.

88. The cardiopulmonary resuscitation monitoring device according to claim 87, wherein said hub comprises means in which to recharge various components of said device requiring power.

89. The cardiopulmonary resuscitation monitoring device according to claim 88, wherein said device further comprising means in which to connect the various components of the claimed device to the means contained in the hub capable of recharging said various components.

90. The cardiopulmonary resuscitation monitoring device according to claim 89, wherein said means to connect the various components of the claimed device to the means contained in the hub capable of recharging said various components comprises one or more wires.

91. The cardiopulmonary resuscitation monitoring device according to claim 89, wherein said means to recharge the various components of the claimed device is capable of recharging said components wirelessly.

92. The cardiopulmonary resuscitation monitoring device according to claim 16, wherein said reference device affixed to the patient in need of cardiopulmonary resuscitation further comprises means in which to record biometrics data from the patient and transmit said data to the processor found within the housing attached to the first responder and/or to an external device.

93. The cardiopulmonary resuscitation monitoring device according to claim 92, wherein said biometrics data transmitted to an external device is used by a treating physician of the patient upon whom cardiopulmonary resuscitation is being performed to allow said treating physician to assess or monitor the health of said patient

94. The cardiopulmonary resuscitation monitoring device according to claim 92, wherein device is designed to be worn on one or more fingers of the first responder performing cardiopulmonary resuscitation on the patient in need of said cardiopulmonary resuscitation.

95. The cardiopulmonary resuscitation monitoring device according to claim 94, wherein said device is designed to be worn on one or more fingers of the first responder and is used to perform cardiopulmonary resuscitation on an infant or small child.

96. The cardiopulmonary resuscitation monitoring device according to claim 9, wherein said device further comprises programming that causes said device to perform as a metronome.

97. The cardiopulmonary resuscitation monitoring device according to claim 96, wherein said first responder matches the compression administered to the patient to the beat of the metronome function of the device.

98. The cardiopulmonary resuscitation monitoring device according to claim 97, further comprising one or more speakers capable of transmitting a sound for each beat of the metronome function of the device.

99. The cardiopulmonary resuscitation monitoring device according to claim 98, wherein said sound is a beep or buzz sound.

100. The cardiopulmonary resuscitation monitoring device according to claim 97, further comprising one or more vibration motors capable of vibrating for each beat of the metronome function of the device.

101. The cardiopulmonary resuscitation monitoring device according to claim 97, further comprising a bulb that turns on and off quickly for each beat of the metronome function of the device.

102. The cardiopulmonary resuscitation monitoring device according to claim 1, further comprising software that suppresses output data when a first responder is not administering chest compressions to a patient upon whom cardiopulmonary resuscitation is being performed.

103. The cardiopulmonary resuscitation monitoring device according to claim 63, wherein said input physical characteristics are used by said device to determine target depths for compressions and target recoil heights to achieve optimal treatment for said patient upon whom cardiopulmonary resuscitation is being performed.

104. The cardiopulmonary resuscitation monitoring device according to claim 103, wherein said device only provides feedback to the first responder when said target depths for compressions and said target recoil heights are not achieved.

105. The cardiopulmonary resuscitation monitoring device according to claim 1, wherein said devise is designed to be used for the training of a first responder.

106. The cardiopulmonary resuscitation monitoring device according to claim 1, wherein said device is designed to be used with an automated external defibrillator.

107. The cardiopulmonary resuscitation monitoring device according to claim 106, wherein said defibrillator comprises pads that adhere to the chest of a patient in need of defibrillation.

108. The cardiopulmonary resuscitation monitoring device according to claim 107, wherein said pads are accelerometers.

109. The cardiopulmonary resuscitation monitoring device according to claim 1, further comprising a gyroscope.

110. The cardiopulmonary resuscitation monitoring device according to claim 14, wherein said visual feedback interface is designed to be read by first responder who is colorblind.

111. The cardiopulmonary resuscitation monitoring device according to claim 1, further comprising a sensor that measures the temperature of the patient upon whom cardiopulmonary resuscitation is being performed.

112. The cardiopulmonary resuscitation monitoring device according to claim 1, further comprising means in which to indicate the time elapsed since cardiopulmonary resuscitation was first administered to said patient in need of cardiopulmonary resuscitation.

113. The cardiopulmonary resuscitation monitoring device according to claim 1, further comprising programming to record the history or the chest compressions performed on the patient and display said history on said visual feedback interface.

114. The cardiopulmonary resuscitation monitoring device according to claim 1, further comprising means in which said device can interact with a secondary device.

115. The cardiopulmonary resuscitation monitoring device according to claim 114, wherein data collected from said secondary device is displayed on the visual feedback interface.

115. The cardiopulmonary resuscitation monitoring device according to any one of claims 14-113, wherein said visual feedback interface is a continuous monitor display.

116. A kit comprising a storage container or hub;
the cardiopulmonary resuscitation monitoring device according to any one of claims 1-114;
optionally one or more batteries; and
one or more disposable adhesive devices;
wherein said storage container or hub contains said cardiopulmonary resuscitation monitoring device; and
further wherein said storage container or hub is formed with a slot in which to store said disposable adhesive devices.

117. The cardiopulmonary resuscitation monitoring device according to claim 14, wherein said device gives instructions before CPR begins.

118. The cardiopulmonary resuscitation monitoring device according to claim 117, wherein said instructions instruct a first responder to notify emergency services.

119. The cardiopulmonary resuscitation monitoring device according to claim 117, wherein said instructions advise a first responder about how to use the device.

120. The cardiopulmonary resuscitation monitoring device according to claim 117, wherein said device is programmed to detect when chest compressions begin.

121. The cardiopulmonary resuscitation monitoring device according to claim 120, wherein said device switches from giving instructions to giving feedback when chest compressions are detected.

122. The cardiopulmonary resuscitation monitoring device according to claim 14, wherein said device is programmed to count and display chest compressions.

123. The cardiopulmonary resuscitation monitoring device according to claim 14, wherein said device is programmed to record elapsed time and display.

124. A method of using a cardiopulmonary resuscitation monitoring device including the steps:
identifying a patient in need cardiopulmonary resuscitation that includes chest compressions;
activating the cardiopulmonary resuscitation monitoring device according to claim any one of claims 1-114;
commencing cardiopulmonary resuscitation on said patient in need thereof;
adjusting the chest compressions based on feedback from the cardiopulmonary resuscitation monitoring device.

125. A method of using a cardiopulmonary resuscitation monitoring device according to claim 124, further comprising the additional step:
receiving instructions from an independent entity or third person that is receiving data from the cardiopulmonary resuscitation monitoring device.

126. The method according to claim 124, wherein the method is being performed by a student practicing cardiopulmonary resuscitation.

127. The method according to claim 126, wherein the student is practicing cardiopulmonary resuscitation on a mannequin.

128. A method of using a cardiopulmonary resuscitation monitoring device comprising the steps of:
activating the device and software;
either holding said system in their hand, attaching it to their hand, or putting the system under their hand;
commencing cardiopulmonary resuscitation on a mannequin;
receiving feedback about the student's cardiopulmonary resuscitation technique from said cardiopulmonary resuscitation monitoring device;
responding to said feedback by modifying the student's cardiopulmonary resuscitation technique;
completing said cardiopulmonary resuscitation training session.

129. The method of using a cardiopulmonary resuscitation monitoring device according to claim 128, comprising the additional step:
reviewing instructional content about said cardiopulmonary resuscitation monitoring device before and/or after the activation step.

130. The method according to claim 129, wherein said instructional content is uploaded onto a separate device.

131. The method according to claim 130, wherein said instructional content comprises a video or a series of videos and/or audio-visual recordings.

132. The method according to claim 130, wherein said instructional content comprises an image or series of images.

133. The method according to claim 130, wherein said instructional content comprises text.

134. The method of using a cardiopulmonary resuscitation monitoring device according to claim 129, comprising the additional step:
answering one or more quiz questions.

135. The method of using a cardiopulmonary resuscitation monitoring device according to 126, wherein the student is practicing cardiopulmonary resuscitation on a mattress, pillow, cushion or a compressible object.

136. The method of using a cardiopulmonary resuscitation monitoring device according to claim 124, wherein said cardiopulmonary resuscitation is performed in mid-air.

137. The method of using a cardiopulmonary resuscitation monitoring device according to claim 126, wherein an instructor teaches the student the steps of the method.

138. The method of using a cardiopulmonary resuscitation monitoring device according to claim 137, wherein said instructor teaches the student by electronic communication.

139. The method of using a cardiopulmonary resuscitation monitoring device according to claim 138, wherein said electronic communication comprises video and audio recordings.
